SEQUENCE LISTING (1 sequence)

SEQ ID NO: 1 - protein, 1189 aa, Homo sapiens
MVPCWNHGNITRSKAEELLSRTGKDGSFLVRASESISRAYALCVLYRNCVYTYRILPNEDDKFTVQASEGVSMRFFTKLDQLIEFYKKENMGLVTHLQYPVPLEEEDTGDDPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEIINPNYMGVGPFGPPMPLHVKQTLSPDQQPTAWSYDQPPKDSPLGPCRGESPPTPPGQPPISPKKFLPSTANRGLPPRTQESRPSDLGKNAGDTLPQEDLPLTKPEMFENPLYGSLSSFPKPAPRKDQESPKMPRKEPPPCPEPGILSPSIVLTKAQEADRGEGPGKQVPAPRLRSFTCSSSAEGRAAGGDKSQGKPKTPVSSQAPVPAKRPIKPSRSEINQQTPPTPTPRPPLPVKSPAVLHLQHSKGRDYRDNTELPHHGKHRPEEGPPGPLGRTAMQ

(19) United States
(12) Patent Application Publication
(10) Pub. No.: US 2014/0287943 A1
(43) Pub. Date: Sep. 25, 2014

(54) ASSAY FOR SHIP1 EXPRESSION, ACTIVITY AND SEQUENCE ALTERATIONS AS A PREDICTOR OF INFLAMMATORY BOWEL DISEASE RISK

(71) Applicants: William G. Kerr, Syracuse, NY (US); Gwenny M. Fuhler, Rotterdam (NL); Sandra Fernandes, Constantia, NY (US)

(72) Inventors: William G. Kerr, Syracuse, NY (US); Gwenny M. Fuhler, Rotterdam (NL); Sandra Fernandes, Constantia, NY (US)

(73) Assignees: ERASMUS UNIVERSITY MEDICAL CENTER ROTTERDAM, Rotterdam (NL); THE RESEARCH FOUNDATION FOR THE STATE UNIVERSITY OF NEW YORK, Albany, NY (US)

(21) Appl. No.: 14/114,015

(22) PCT Filed: Apr. 27, 2012
(86) PCT No.: PCT/US2012/035461

§ 371 Date: Jun. 12, 2014

Related U.S. Application Data
(60) Provisional application No. 61/479,597, filed on Apr. 27, 2011.

(51) Int. Cl. C12Q 1/42; G01N 33/573; C12Q 1/68

(52) U.S. Cl. CPC C12Q 1/42; C12Q 1/6883; G01N 33/573
USPC 506/9; 435/21; 435/6.11; 435/7.4; 435/6.12

(57) ABSTRACT

The present disclosure is directed to detecting colon disorders by measuring the expression of SHIP1 in a sample of PBMCs. One method includes the following steps, obtaining a sample including peripheral blood mononuclear cells (PBMCs) from a subject and determining whether SHIP1 is underexpressed in the PBMCs or lacks normal enzymatic activity. The present disclosure is also directed to a method of determining the expression of SHIP1 protein expression and SHIP1 enzyme activity in PBMCs. This method includes the following steps, obtaining a sample comprising PBMCs from a subject and determining the amount of SHIP1 in the PBMCs.

### CROSS REFERENCE TO RELATED APPLICATION

This application claims the benefit of priority of U.S. Provisional Patent Application No. 61/479,597, filed Apr. 27, 2011, the entire contents of which are incorporated herein by reference.

### FIELD OF THE DISCLOSURE

The disclosure relates generally to the field of detecting colon disorders. More specifically, the present disclosure is directed to detecting colon disorders by measuring the expression and activity of SH2-containing inositol-5-phosphatase 1 (SHIP1) in a sample of peripheral blood mononuclear cells (PBMCs).

### BACKGROUND OF THE DISCLOSURE

Crohn's Disease (CD) is one example of a chronic, relapsing idiopathic inflammatory bowel disease (IBD) that can affect various sites within the gastrointestinal tract, classically in the ileum. IBD is a group of inflammatory conditions, including CD, in the colon and small intestine of mammals as well as ulcerative colitis (UC). IBDs lead to abdominal cramping, diarrhea and gastrointestinal bleeding.

CD histopathological lesions begin with the formation of multiple aphthoid ulcers infiltrated by neutrophils that progress and coalesce into delimited, regional, transmural inflammations with granuloma formation, thickening of the muscularis propria, strictures, fissures and fistulas.

Although genome-wide association studies (GWAS) have identified more than 30 susceptible genetic loci for IBD that may disturb either intestinal epithelial cell (IEC) barrier homeostasis or immune effector cell functions and interactions with luminal flora and antigens, many susceptibility genes possess differing functions in IEC and hematopoietic cells. Therefore, primary IEC or immunological defects in genetically susceptible individuals often remain a mystery.

An accurate way to determine whether an individual has an increased probability of developing CD or other forms of IBD is desired.

Embodiments of the present application provide a process that addresses the above and other issues.

### SUMMARY OF THE DISCLOSURE

The present disclosure is directed to detecting colon disorders by measuring the expression of SH2-containing inositol-5-phosphatase 1 (SHIP1) in a sample of peripheral blood mononuclear cells (PBMCs). This method includes the following steps, obtaining a sample (including PBMCs) from a subject and determining whether SHIP1 is underexpressed in the PBMCs.

The present disclosure is also directed to a method of determining the expression of SHIP1 protein expression or activity in PBMCs. This method includes the following steps, obtaining a sample comprising PBMCs from a subject and determining the amount of SHIP-1 in the PBMCs.

### BRIEF DESCRIPTION OF THE DRAWINGS

The present disclosure will be better understood by reference to the following drawings of which:

FIG.1 illustrates SHIP1 expression based on samples with differing amounts of total cell protein.

FIG. 2 illustrates SHIP1 expression based on samples with differing amounts of total cell protein.

FIG. 3 illustrates the determination of the quantitative range of the malachite green assay.

FIG. 4 illustrates SHIP1 expression based on samples with differing amounts of total cell protein.

FIG. 5 illustrates SHIP1 expression based on samples with differing immunoprecipitants.

FIG. 6 illustrates the determination of the quantitative range of the malachite green assay.

FIG. 7 is illustrative of two experiments to determine varying SHIP1 activity between subjects with Crohn's Disease and subjects that did not have Crohn's Disease.

FIG. 8 is illustrative of an experiment to determine which Single Nucleotide Polymorphism (SNP) is indicative of idiopathic inflammatory bowel disease (IBD).

### DETAILED DESCRIPTION OF THE DISCLOSURE

The methods of the present disclosure provide improvements over traditional colon disorder detection methods which do not quantify SH2-containing inositol-5-phosphatase 1 (SHIP1) expression and enzyme activity. The improvements are, inter alia, a single test that determines SHIP l′s involvement in colon disorders to allow for more efficacious treatment of the colon disorder.

One method of the present application includes a method of detecting a colon disorder in a subject containing several steps. Initially a sample including peripheral blood mononuclear cells (PBMCs) is taken from the subject. The subject can be any mammal, including humans. The sample taken can be any sample from the subject containing PBMCs, including a whole blood sample. The sample can also be cells from the subject that are cultured in vitro. Appropriate isolation steps may be taken, and/or pretreatments carried out to determine SHIP1 expression in the sample. The sample may be preserved or pre-treated, or prepared for histological and immunohistochemical analysis. Red blood cells can be lysed by treatment with hypotonic solutions from nucleated cells, and separation can be achieved by differential centrifugation and other methods known in the art. For example, a Ficoll step gradient procedure can be utilized. In this example, PBMC-enriched cell populations can be obtained using the buffy coat method. Samples may be cryopreserved prior to determination of SHIP1 expression.

The colon disorder being detected can include inflammatory bowel diseases such as Crohn's Disease (CD) and ulcerative colitis among others. This method also has an advantage in that it may be used to test for colon disorders before a more invasive test for colon disorders is used. Optionally, the method could further comprise carrying out at least one confirmatory test including but not limited to, a blood test, test for blood in a stool sample, test for a target microorganism in a stool sample, colonoscopy, sigmoidoscopy, X-ray with barium, computerized axial tomography, capsule tomography and combinations thereof.

The next step of the method includes a determination of whether SHIP1 is under expressed in the PBMCs. Under expression of SHIP1 is indicative of a subject having IBDs such as CD, or having an increased chance of contracting IBDs such as CD.

In another method, the expression of SHIP1 protein expression and enzyme activity in PBMCs is determined by first obtaining a sample comprising PBMCs from a subject and then determining the amount of SHIP1 in the PBMCs. The determination of the amount of SHIP1 expression and the amount of SHIP1 enzyme activity in the PBMCs is described below.

SHIP1 expression can be detected in many ways including the use of antibodies to detect the presence of the proteins or by determining the presence and amount of mRNA coding for SHIP1. Gene expression analysis can be performed at the mRNA or protein level to detect differences in SHIP1 gene expression between populations of target cells (e.g., PBMCs) of a subject and reference cells (e.g., an appropriate control) to determine whether or not SHIP1 is being expressed and to what extent. SHIP1 expression can be determined in multiple samples taken at different times in an effort to monitor SHIP1 expression.

Further, SHIP1 expression can be detected through the use of immunological reagents. Immunological reagents are intended to cover antisera and antibodies, particularly monoclonal antibodies, as well as fragments thereof that bind to SHIP1. Also included under the term immunological reagents are chimeric antibodies, humanized antibodies, recombinantly-produced antibodies, and fragments thereof, as well as aptamers (i.e., oligonucleotides) capable of interacting with target molecules such as peptides. Immunological methods used in conjunction with reagents include direct and indirect (for example, sandwich type) labeling techniques, immunoaffinity columns, immunomagnetic beads, fluorescence activated cell sorting (FACS), enzyme-linked immunosorbent assays (ELISA) and radioimmune assays (RIA). For use in these assays, the immunological reagents can be labeled, using fluorescence, antigenic, radioisotopic or biotin labels, among others, or a labeled secondary or tertiary immunological detection reagent can be used to detect binding of the immunological reagents used in determining the presence of SHIP1.

Another example of assays described above is a bead based assay. Any suitable bead based assay can be used, as an example, LUMINEX® beads or other fluorescent beads, or beads varying in light scattering parameters can be conjugated to target parameters including antibodies to SHIP1 and conjugated proteins that interact specifically with SHIP1, its substrate, a non-hydrolyzable version of its substrate or other small molecules that bind to SHIP1 specifically. The conjugated beads are added to the sample, sample lysate, or to the removed supernatant, allowing bead binding to target parameters. Also, fluorescent antibody to a distinct epitope of the target parameter can be used to measure the level of target parameter bound. The fluorescence and light scatter characteristics of the beads constitute an identifier of the target parameter, and fluorescence derived from added antibody to the target parameter is an indication of the quantity of target parameter bound, and hence a readout of the individual parameter.

Flow cytometry may be used to quantitate parameters such as the presence of cell surface proteins or conformational or posttranslational modification thereof; intracellular or secreted protein, where permeabilization allows antibody (or probe) access, and the like. The readouts of selected parameters are capable of being read simultaneously, or in sequence during a single analysis, as for example through the use of fluorescent antibodies to cell surface molecules or to intracellular proteins after fixation and permeabilization of cells (NB: SHIP is an intracellular protein). As an example, these can be tagged with different fluorochromes, fluorescent bead, tags, e.g. quantum dots, etc., allowing analysis of up to 4 or more fluorescent colors simultaneously by flow cytometry. Data gathered from the flow cytometry process is then used to determine the expression value of SHIP1 from the sample taken from the patient.

The expression of SHIP1 at the mRNA level can also be determined using nucleic acid hybridization techniques and associated hybridization methods to detect the presence of mRNA with a sampled cell. For example, a nucleic acid that is complementary to and hybridizes under stringent conditions to the mRNA of a portion of SHIP1 can be detectably labeled. This detectably labeled nucleic acid can be contacted with a cell or an extract of a cell to detect the presence and amount of the mRNA that encodes SHIP1. The amount of nucleic acids that encode SHIP1 is assumed to correlate with the expression of the SHIP1 in a sample cell. Further, RNA based detection of SHIP1 expression assumes there is no translational or post-translation regulation of SHIP protein levels. The selection of an appropriate nucleic acid molecule for use as a probe can be made by studying the nucleic acid sequences of SHIP1 and determining an appropriate length. A unique sequence should be determined that selectively hybridizes under stringent conditions to the mRNA of SHIP1.

The expression of SHIP1 can also be determined using a polymerase chain reaction (PCR). PCR allows for the rapid generation of multiple copies of DNA sequences by providing 5′ and 3′ primers that hybridize to sequences present in an RNA or DNA molecule, and further providing free nucleotides and an enzyme which fills in the complementary bases to the nucleotide sequence between the primers with the free nucleotides to produce a complementary strand of DNA. The enzyme will fill in the complementary sequences adjacent to the primers. If both the 5′ primer and 3′ primer hybridize to nucleotide sequences on the same small fragment of nucleic acid, exponential amplification of a specific double-stranded size product results. If only a single primer hybridizes to the nucleic acid fragment, linear amplification produces single-stranded products of variable length.

PCR primers can be designed by a skilled artisan using sequence information. The nucleotide sequences of SHIP1 transcripts are known. When performing PCR on extracted mRNA or cDNA generate therefrom, if the SHIP1 transcript of cDNA generated therefrom is present, multiple copies of the mRNA or cDNA will be made. If it is not present, PCR will not generate a discrete detectable product. Primers are generally 8-50 nucleotides, which are identical or complementary to and therefore hybridize to the SHIP1 transcript or cDNA generated therefrom.

mRNA or cDNA is combined with the primers, free nucleotides and enzyme following standard PCR protocols. The mixture undergoes a series of temperature changes. If the SHIP1 transcript of cDNA generated therefrom is present, that is if both primers hybridize to sequences on the same molecule, the molecule comprising the primers and the intervening complementary sequences will be exponentially amplified. The amplified DNA can be detected by a variety of means. If no SHIP1 transcript of cDNA generated therefrom is present, no PCR product will be exponentially amplified. The PCR technology therefore provides a reliable method of detecting the SHIP1 transcript in a sample.

Gene expression analysis can be also be performed to detect differences in gene expression between populations of sampled cells of a subject and control cells to determine whether or not SHIP1 is being expressed and to what extent. Hybridization of gene expression microarrays can be used to produce patterns of gene expression of SHIP1. Identification of genes and patterns of genes differentially expressed in sample cells can be established by comparison of the gene expression pattern obtained by performing the microarray hybridization analysis on cDNA from target cells in comparison to that of control cells.

According to the present claims, a colon disorder can be detected by determining whether or not SHIP1 is expressed in a tissue type, such as PBMCs or blood, that normally express SHIP1, and to what extent. According to one of the present methods, underexpression of SHIP1 is indicative of the colon disorder and is indicative of a subject having IBDs such as CD, or having an increased chance of contracting IBDs such as CD.

To determine whether a subject's measured expression is an underexpression, the measured expression is compared to a suitable control level that is associated with normal SHIP1 expression. This suitable control level can take many forms that include, but are not limited to, a transcription rate mRNA level, translation rate, protein level, protein structure, biological activity, cellular characteristic or property, genotype, phenotype and enzymatic activity associated with SHIP1. In one embodiment, a suitable control is a predetermined SHIP1 level that is compared to an SHIP1 level in a sample obtained from a subject being identified as having or not having a colon disorder (control sample). In another embodiment, a suitable control level is determined as a level of expression among a population of individuals that do not have a colon disorder. In another embodiment, a suitable control can be a predetermined SHIP1 level, which is compared to an SHIP1 level in a sample taken from a subject in which a clinical measure was achieved, for example an SHIP1 level obtained from a subject who has been previously treated for a colon disorder.

In another embodiment, to determine whether a subject's measured expression is an underexpression, the measured expression can be compared to an internal standard in a subject like other unrelated genes that are expressed at constant levels from subject to subject. Some examples of these genes that are expressed at constant levels include beta-actin, Glyceraldehyde 3-phosphate dehydrogenase (GAPDH) and beta-tubulin.

In another embodiment, a suitable control can be a single cut-off value, such as a median or mean. A single cut-off value can be established, for example, based upon a mean of an SHIP1 level or activity in samples (in controls having a normal SHIP1 level or activity) that correlate with lack of or resistance to a colon disorder. In practice, the determination of SHIP1 may be qualitative, quantitative or semi-quantitative. In one embodiment, a quantitative value of SHIP1 level or activity is determined that can be compared to that of an appropriate control. In another embodiment, a reference or standard curve with different amounts of recombinant SHIP1 can be used. The reference or standard curve generated could be used to determine the absolute amount of enzymatically active SHIP and SHIP protein on a per cell basis in a given PBMC lysate from a subject.

The disclosure also includes another method for detecting a colon disorder in a subject, the method includes a first step of obtaining a sample from the subject containing the gene phosphatidylinositol-3,4,5-trisphosphate 5-phosphatase 1(INPP5D). This sample can be obtained in any suitable way, such as withdrawing a blood sample using a hypodermic needle and the subject can be any mammal, including a human.

Next, a determination is made as to whether the INPP5D gene present in the sample includes a Single Nucleotide Polymorphism (SNP). The SNP is indicative of a colon disorder, which can be any colon disorder including inflammatory bowel disease and ulcerative colitis. The SNP can be selected from the group consisting of a G:A SNP at chromosome position 233,924,659 of a promoter exon, a T:C SNP at chromosome position 233,924,923 of the promoter exon, a T:C SNP at chromosome position 234,055,004 of exon 6_{—}7 and a G:A SNP at chromosome position 234,107.

Once the presence of one or more SNPs has been determined, the subject is assigned to either of two groups, a colon disorder group or a healthy group. In this context, healthy is meant to mean a subject that does not have a colon disorder. A subject is assigned to the colon disorder group if their sample includes one or more SNPs. This assignment indicates that the subject has a higher probability of having a colon disorder as compared to a subject that does not have any SNPs. A subject is assigned to a healthy group if their sample does not include any SNPs. This assignment indicates that the subject has a lower probability of having a colon disorder as compared to a subject that does have SNPs.

The present disclosure is further illustrated by the following non-limiting examples.

### EXAMPLES

### Example 1

One example of a method of determining the expression of SH2-containing inositol-5-phosphatase 1 (SHIP1) protein expression was performed and is described below. Peripheral blood mononuclear cells (PBMCs) were isolated from heparin anti-coagulated blood by Ficoll density gradient centrifugation. Cells were washed twice, and re-suspended in IP-lysis buffer (20 mM Tris pH 7.5, 150 mM NaCl, 1 mM EDTA, 1 mM EGTA, 1% Triton X100) containing PMSF and Halt protease inhibitors. After lysis on ice for 10 minutes, lysates were cleared by centrifugation for 10 minutes. Protein concentration in the supernatants was determined by RC DC protein assay (Biorad).

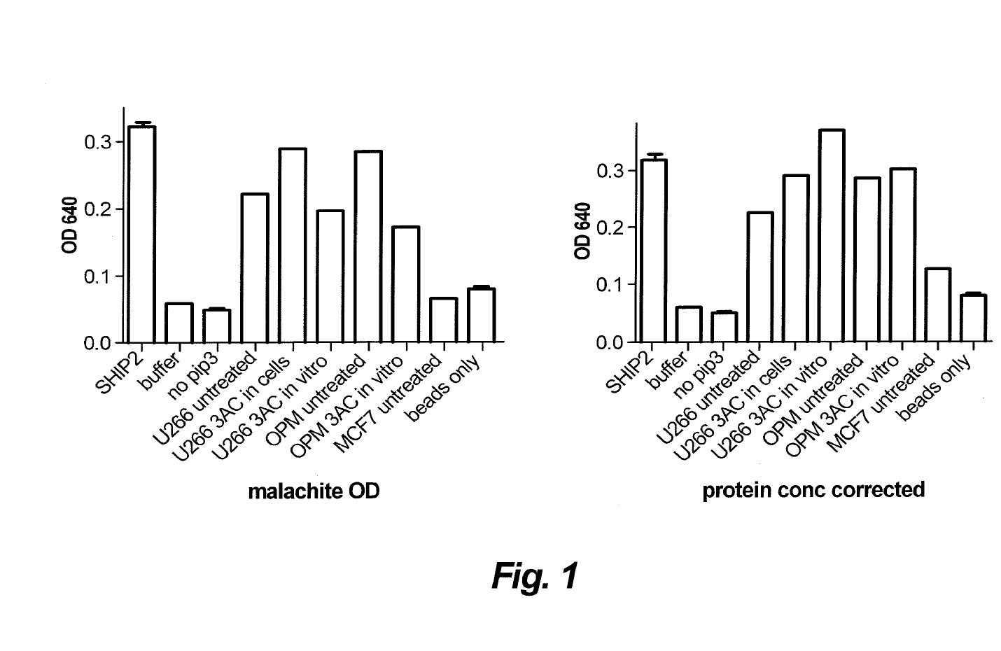

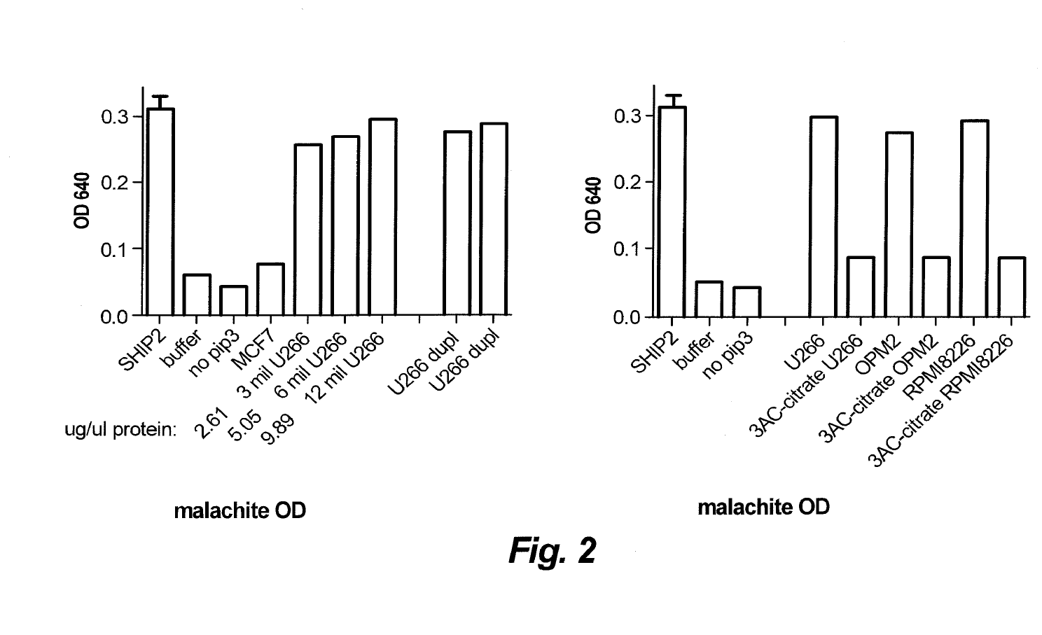

Lysate containing 300 μg protein was transferred to a new Eppendorf vessel, and volume was adjusted to 300 μL with IP-lysis buffer. Although 300 μg of protein was utilized in this example, as can be seen from FIGS. 1 and 2, protein levels as low as 25 μg can be used to determine SHIP1 expression. Lysates were pre-cleared with 25 μL pre-washed Gammabind G Sepharose beads for 1 hour. Supernatant was transferred to a new Eppendorf vessel, and 3 μL anti-SHIP1 antibody (P1C1, Santa Cruz) was added. Samples were rotated at 4° C. overnight, after which 50 μL pre-washed Gammabind G Sepharose beads were added for an additional 2 hours.

Beads were washed 4 times with IP-lysis buffer, and 1 time with malachite buffer (TBS, 10 mM MgCl₂). Beads were re-suspended in 25 μL malachite buffer. PIP3 (120 μM) was added and mixtures were incubated for 35 minutes at room temperature. Supernatants were transferred to 96 wells plate, 100 μL, malachite green was added and OD620 was measured after 20 minutes of incubation at room temperature in the dark.

In the left panel of FIG. 1, SHIP1 was precipitated from cell lines that were untreated, or treated with 3AC, after which isolated SHIP1 was subjected to malachite green assay. In addition, SHIP1 was precipitated from untreated cells, and treated with 3AC for 5 minutes prior to performing malachite assay. Recombinant SHIP2 was used as positive control for the malachite green assay. Negative controls included buffer with PIP3 but no SHIP immunoprecipitate (buffer), buffer without PIP3 or SHIP immunoprecipitate (no pip3) and beads with PIP3 (beads only). In the right panel of FIG. 1, the SHIP activity was corrected for the amount of protein added as determined by RC/DC protein kit. This correction was done to account for the amount of protein added to each reaction that was not equalized prior to the precipitation.

For the other samples shown in FIG. 1, total protein concentration was determined by RC/DC kit from Biorad. SHIP1 protein expression was quantitatively determined by Western Blot analysis using the Odyssey infrared imaging System. The other measured samples were chosen because of their known expression of SHIP1. MCF-1 cells were chosen as negative control, as they express SHIP2, but not SHIP1. These other results allow for the correlation of SHIP1 protein levels to total protein as measured by RC/DC kit, actin protein levels (a commonly used household protein to determine equal loading of western blots) or SHIP1 activity as determined by phosphatase assay.

For the following figures in Example 1, described below, similar procedures were practiced as were described for FIG. 1. Variations from the procedure used to generate FIG. 1 are described below as they pertain to the subsequent figures.

FIG. 2 illustrates SHIP1 expression based on samples with differing amounts of total cell protein. As can be seen in the left panel of FIG. 2, SHIP1 was precipitated from different amounts of U266 cells (3, 6, 12x10e6) and malachite green assay was performed. Protein quantification by RC/DC kit shows a correlation to the amount of cells used, however, the malachite green measured does not correlate as well. This result is indicative of the measurements being at the top end of the detection level of the assay.

In the right panel of FIG. 2, SHIP1 was immunoprecipitated from different cell lines, after which the precipitate was treated with 3AC or not for 2 minutes and malachite green assay was performed.

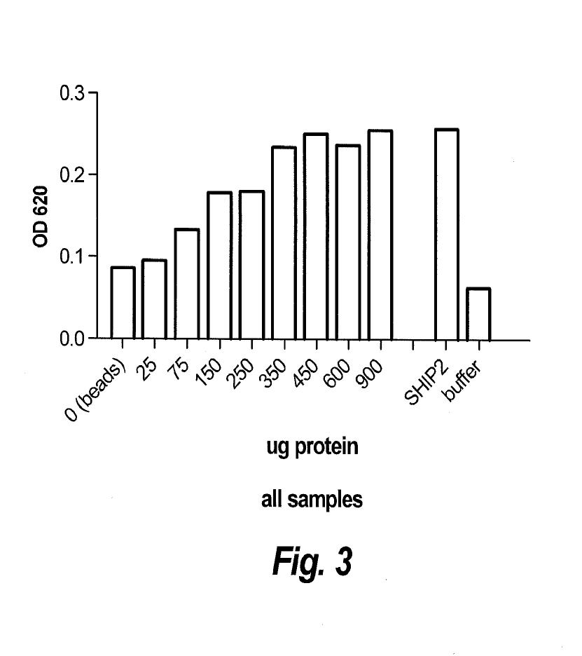

In FIG. 3, to determine the quantitative range of the assay, SHIP1 was precipitated from increasing concentrations of protein, a lysate made of OPM2 cells was used. As shown in FIG. 3, at the higher end of the concentration curve, no additional increase in SHIP1 activity is detected. This is indicative of the quantitative range for the malachite green assay ranging from 25 to 350 μg of protein for OPM2 cells.

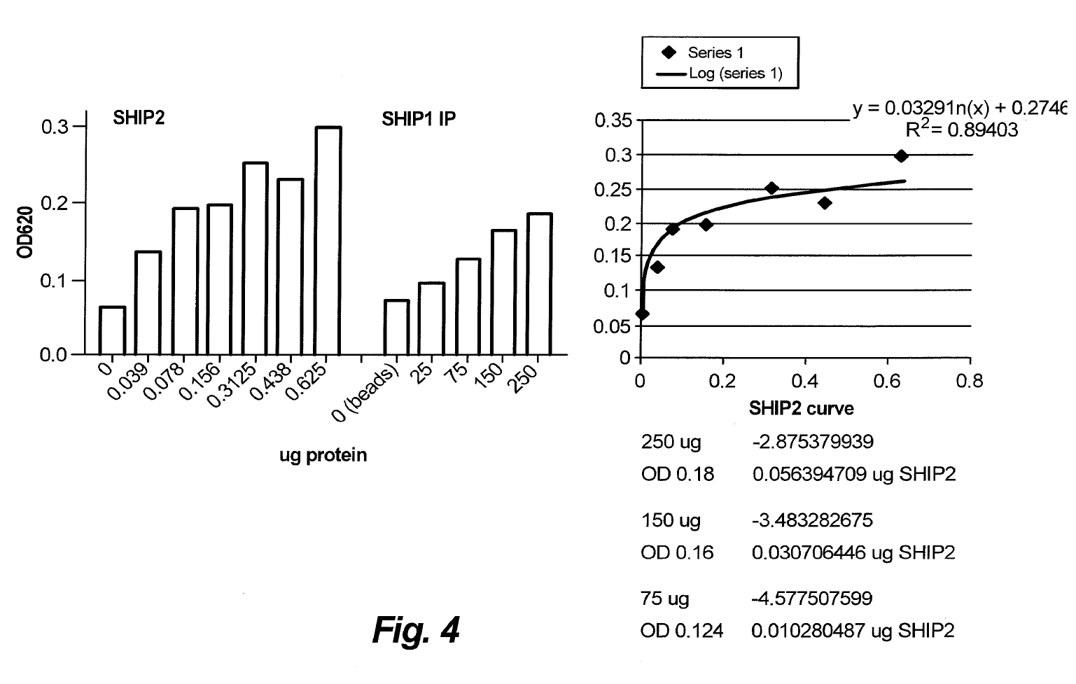

As seen in FIG. 4, increasing concentrations of protein (OPM2 lysate) were subjected to SHIP1 immunoprecipitation and malachite green assay. In addition, increasing concentration of recombinant SHIP2 were added to the malachite green assay. Plotting the concentration SHIP2 protein against the OD (Optical Density (absorbance)) a description of the curve was made, that was then used to determine the approximate amount of SHIP1 present in the OPM2 lysates. This data is based on the assumption that SHIP1 and SHIP2 have equal efficiency rates in the malachite green assay.

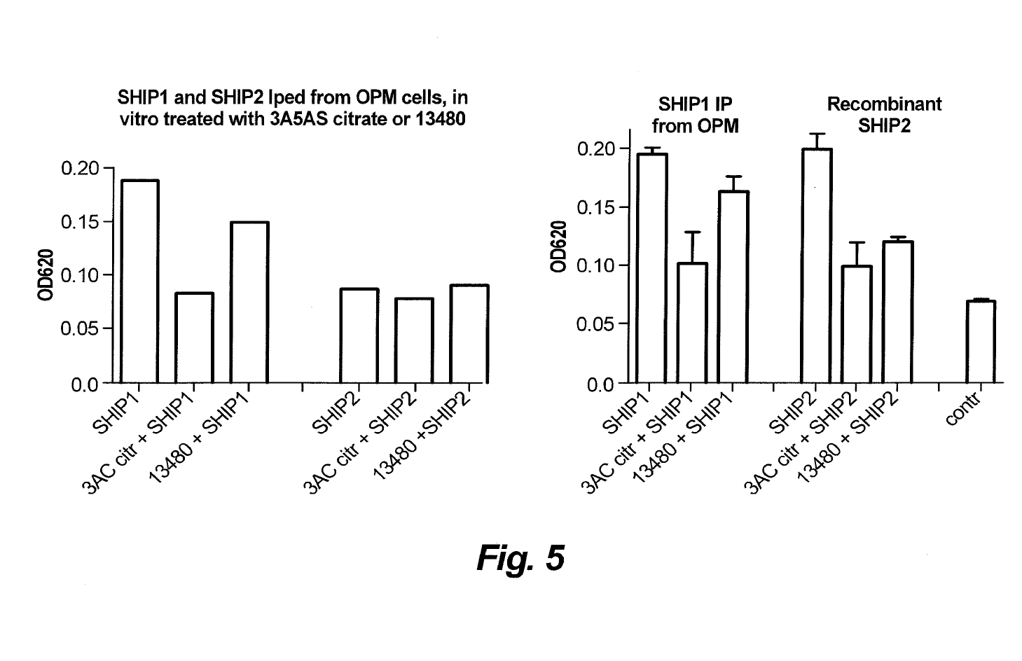

As seen in the left panel of FIG. 5, SHIP1 and SHIP2 were immunoprecipitated from OPM2 cell lysates, after which they were treated for 2 minutes with either 3AC-citrate, 13480 (pan-SHIP inhibitor) or DMSO (dimethyl sulfoxide(vehicle control)). SHIP2 precipitation was not efficient, as no malachite green was detected. The right panel of FIG. 5 shows a mean of 2 experiments in which SHIP1 was precipitated from OPM2 cells and treated with 3AC-citrate, 13480 or DMSO before being subjected to malachite green assay. In addition, recombinant SHIP2 was also treated with these compounds and subjected to malachite green assay.

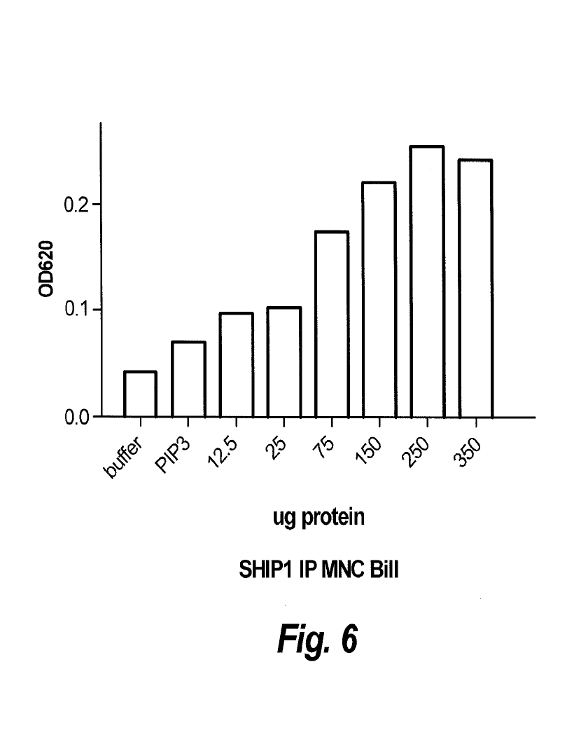

As shown in FIG. 6, the quantitative range of PMN for this assay runs from 12.5 to 250 μg of protein. To determine this range, mononuclear cell fraction from peripheral blood of healthy control was isolated, lysed, and SHIP1 was precipitated from increasing concentrations of protein. The quantitative range of the assay may determine the cell type used, as the concentration of SHIP1 in different tissues may be different.

### Example 2

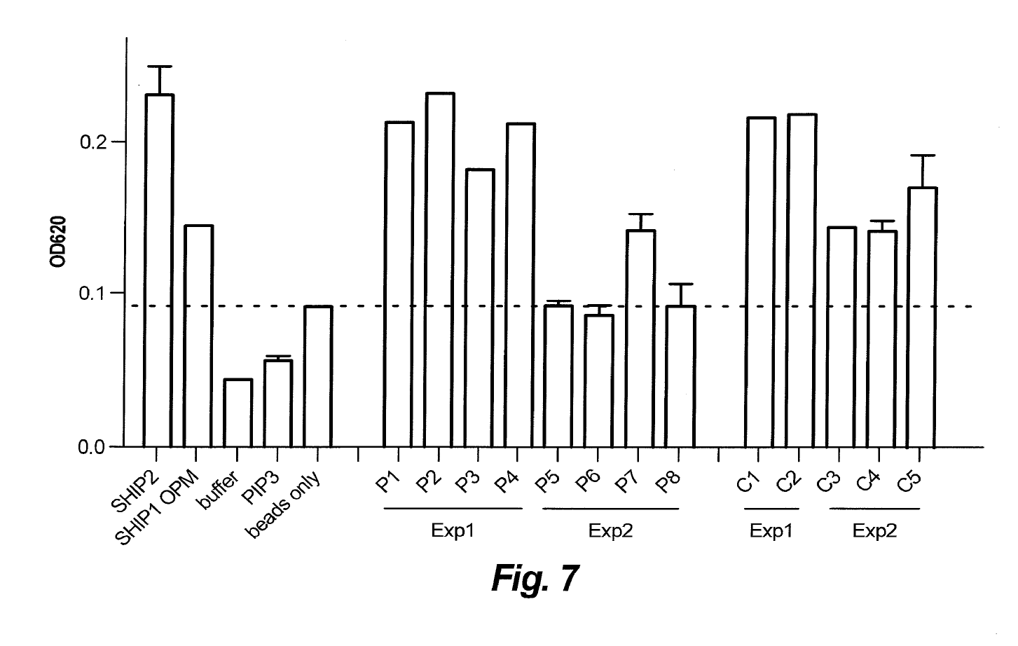

In FIG. 7, mononuclear cell fractions from peripheral blood of healthy controls (C1 to 5) and Crohn's patients (P1 to P8) were isolated in two experiments (exp 1 and exp2). SHIP1 was precipitated from 200 μg of protein, and SHIP1 activity was determined by malachite green assay. SHIP1 precipitated from OPM2 lysate and recombinant SHIP2 were used as positive controls. Negative controls included buffer with PIP3 but no SHIP1 immunoprecipitate (buffer), buffer without PIP3 or SHIP1 immunoprecipitate (no pip3) and beads with PIP3 (beads only). As especially can be seen in the comparison of subjects in experiment 2, P5-P8 for Crohn's patients and C3-C5 for healthy controls, SHIP1 activity is less in the Crohn's patients than in the healthy controls.

The dashed line in FIG. 7 represents the level of activity of “beads only ” which indicates the background activity. This dashed line is indicative of SHIP1 activity not reaching values above background in some subjects.

### Example 3

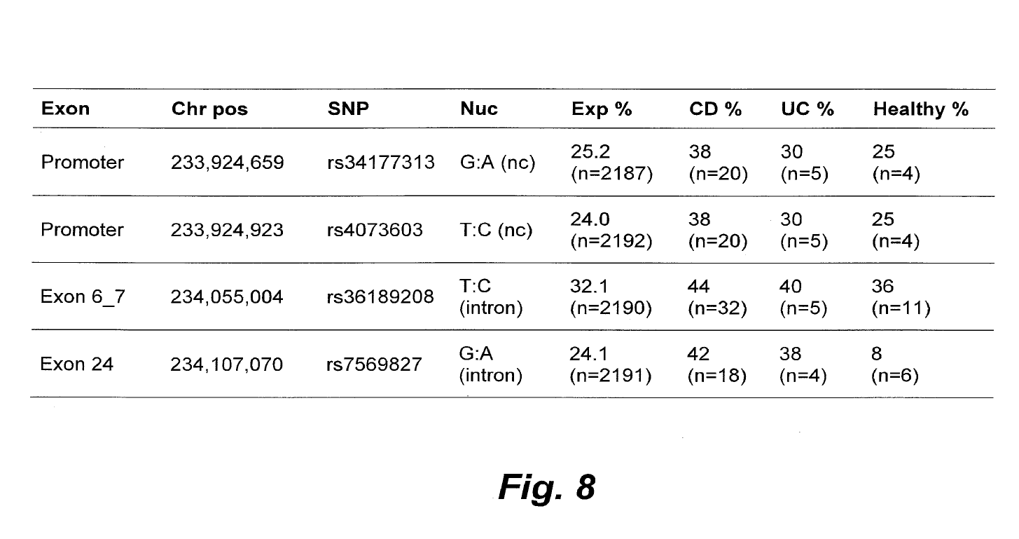

As shown in FIG. 8, a statistical analysis of a number of Single Nucleotide Polymorphisms (SNP) was done to determine whether or not there are any SNPs that are indicative of any colon disorders. SNPs on the gene phosphatidylinositol-3,4,5-trisphosphate 5-phosphatase 1(INPP5D), which codes for SH domain-containing inositol-5′-phosphatase 1 (SHIP-1), were analyzed. Expression of this protein occurs in hematopoietic cells where its movement from the cytosol to the plasma membrane is mediated by tyrosine phosphorylation. At the plasma membrane, the protein hydrolyzes the 5′ phosphate from phosphatidylinositol (3,4,5)-trisphosphate and inositol-1,3,4,5-tetrakisphosphate, thereby affecting multiple signaling pathways.

As shown in FIG. 8, a number of SNPs at different locations on INPP5D were determined to be indicative of colon disorders. Based on a large set from the SNP database in the University of California Santa Cruz (UCSC) Genome Browser on Human February 2009 (GRCh37/hg19) Assembly, an expected frequency of SNP at a specific position was determined and shown in the Exp% column. This Genome Browser is available through the following website: http://genome.ucsc.edu/cgi-bin/hgGateway. Once the expected frequency of SNPs at a number of positions was determined, a determination was made as to the frequency of the SNP in a number of individuals. For example, in evaluating the first SNP, rs34177313 of FIG. 8, the INPP5D gene of 29 subjects was sequenced. It was determined that 38% of the tested subjects with the specific SNP had Crohn's disease, 30% of the tested subjects with the specific SNP had ulcerative colitis and 25% of the tested subjects with the specific SNP did not have any colon disorders.

What is claimed is:

1. A method of detecting a colon disorder in a subject comprising:
obtaining a sample comprising peripheral blood mononuclear cells (PBMCs) of the subject; and
determining levels of expression or enzymatic activity of SH2-containing inositol-5-phosphatase 1 (SHIP1) in the PBMCs, wherein underexpression or lack of a normal enzymatic activity is indicative of the colon disorder.

2. The method of claim 1, wherein underexpression of SHIP1 is indicative of the colon disorder.

3. The method of claim 1, wherein the subject is a human.

4. The method of claim 1, wherein the colon disorder is inflammatory bowel disease.

5. The method of claim 4, wherein the colon disorder is Crohn's Disease.

6. The method of claim 1, wherein the sample is a whole blood sample.

7. A method of determining the expression of SH2-containing inositol-5-phosphatase 1 (SHIP1) protein expression and enzyme activity in peripheral blood mononuclear cells (PBMCs), comprising:
obtaining a sample comprising PBMCs from a subject;
determining the amount of SHIP-1 in the PBMCs; and
determining the amount of enzyme activity in the PBMCs.

8. A method of detecting a colon disorder in a subject comprising:
obtaining a sample containing the gene phosphatidylinositol-3,4,5-trisphosphate 5-phosphatase 1(INPP5D) of the subject;
determining whether the INPP5D includes a Single Nucleotide Polymorphism (SNP); and
assigning the subject to a colon disorder group or a healthy group, wherein presence of one or more SNPs in the subject will cause the subject to be assigned to the colon disorder group.

9. The method of claim 8, wherein the subject is a human.

10. The method of claim 8, wherein the colon disorder is inflammatory bowel disease.

11. The method of claim 8, wherein the colon disorder is ulcerative colitis.

12. The method of claim 8, wherein the SNP is selected from the group consisting of a G:A SNP at chromosome position 233,924,659 of a promoter exon, a T:C SNP at chromosome position 233,924,923 of the promoter exon, a T:C SNP at chromosome position 234,055,004 of exon 6_{—}7 and a G:A SNP at chromosome position 234,107,070 of exon 24.
